(19) United States
(12) Patent Application Publication
(10) Pub. No.: US 2006/0247615 A1
(43) Pub. Date: Nov. 2, 2006

(54) Multi-element bi-polar ablation electrode

(75) Inventors: Orla McCullagh, Maynard, MA (US); Robert C. Thistle, Bridgewater, MA (US)

Correspondence Address: Vista IP Law Group LLP, 2040 MAIN STREET, 9TH FLOOR, IRVINE, CA, 92614, US

(73) Assignee: Boston Scientific Scimed, Inc., Maple Grove, MN (US)

(21) Appl. No.: 11/118,823

(22) Filed: Apr. 28, 2005

(51) Int. Cl. A61B 18/14

(52) U.S. Cl. 606/41; 606/50; 606/48

(57) ABSTRACT

An electrosurgical system includes an elongate device having a distal end portion carrying a plurality of electrically conductive elements, each element electrically insulated from the other elements. A generator is provided having first and second terminals for forming an electrical circuit. Control circuitry which may be located in the device, the generator or external to both, selectively connects a first electrode comprising one or more of the elements to the first generator terminal, and a second electrode comprising one or more of the further elements to the second generator terminal, to form an electrical circuit that is completed by electrical conduction between the first electrode and the second electrode.

### FIELD OF INVENTION

The present invention relates generally to medical devices for treating tumors and, more specifically, to devices for treating tumors using radiofrequency energy.

### BACKGROUND

Known radiofrequency (“RF”) ablation devices employ an array of electrode tines deployed from the end of a single delivery cannula to transmit RF energy into a targeted tissue area, e.g., a tumor, causing heating and eventual ablation of the tissue area. Such electrode array devices include the LeVeen Needle and Co-Access (collectively “LeVeen Needle”) devices manufactured and distributed by Boston Scientific Corporation.

In use, the distal end of the cannula is positioned in a target tissue region, e.g., a cancerous tumor, and the electrode tines are extended distally and radially outward from the cannula, everting into an “umbrella-like” deployed configuration to envelop the target tissue region. The electrode tines are coupled through a handle of the device to a RF power generator, collectively acting as a first (or “active”) pole of an electrical circuit powered by the generator. The circuit is completed with an external pad (“return”) electrode secured to the patient's skin. The LeVeen Needle products are operated in monopolar mode, which is to say, virtually all of the energy applied through the electrical circuit is dissipated in, and causes necrosis of, the target tissue region proximate the respective electrode tines, which have a relatively tiny surface area compared with the external return pad electrode.

While effective, such electrode array devices are somewhat complex to manufacture and use. Thus, a more simple, “single needle” device may be preferable in some instances. Such single needle devices may also be operated in a monopolar mode, or it may be preferable to provide a pair of electrodes carried on a single needle device that are operated in a bi-polar mode, i.e., wherein the generator circuit is completed between the respective electrodes carried on the device, with the energy dissipated in, and causing necrosis of, the target tissue proximate and between the respective electrodes of the pair.

### SUMMARY OF INVENTION

In an embodiment constructed according to one aspect of the invention, an electrosurgical device includes a proximal handle portion and an elongate member extending distally from the handle portion. A plurality of electrically conductive elements are attached to a distal end of the elongate member. A first electrode including one or more of the conductive elements is electrically connected to a first electrical connector located on the handle portion. A second electrode including one or more further of the conductive elements is electrically connected to a second electrical connector located on the handle portion, wherein the one or more conductive elements of the second electrode are electrically insulated from the one or more conductive elements of the first electrode. The first and second electrical connectors are adapted for connection to respective first (“active”) and second (“return”) terminals of a power source to form an electrical circuit that is completed by electrical conduction between the one or more conductive elements of the first electrode and the one or more conductive elements of the second electrode.

In one embodiment, the first electrode comprises an inner core of conductive elements, and the second electrode comprises an outer ring of conductive elements at least partially surrounding the inner core. In this and/or other embodiments, the conductive element(s) of the first electrode may extend distally beyond the conductive element(s) of the second electrode, or vice versa. In one embodiment, the conductive elements are positionable within a lumen of a delivery cannula, e.g., of an obtuator assembly, for locating the elements in a target tissue region in a body. In this and/or other embodiments, at least some of the conductive elements may be biased to move radially outward in body tissue when the delivery cannula is retracted to expose the elements in the target tissue region. In another embodiment, the conductive elements have sufficient column strength, and are tethered together with sharp tips to collectively form a tissue piercing distal end of the elongate member, thereby assisting the elongate member to be moved directly through solid body tissue for locating the elements in a target tissue region.

In an embodiment constructed in accordance with another aspect of the invention, an electrosurgical system includes an electrosurgical elongate device having a proximal handle portion and an elongate member extending distally from the handle portion. A plurality of electrically conductive elements are attached to a distal end of the elongate member, each element electrically insulated from the other elements. The system includes a generator having first (“active”) and second (“return”) terminals for forming an electrical circuit powered by the generator.

The system further includes control circuitry which selectively electrically connects a first electrode comprising one or more of the conductive elements to the active terminal and a second electrode comprising one or more of the further conductive elements to the return terminal to form an electrical circuit that is completed by electrical conduction between the element(s) of the first electrode and the element(s) of the second electrode. In one embodiment, each conductive element is connected to a respective electrical connector located on the handle portion of the elongate device, and the control circuitry is located external to the elongate device, e.g., in the generator or in an adjunct device. In this embodiment, the control circuitry is configured to electrically connect one of the first and second generator terminals to selected ones of the conductive elements via the respective electrical connectors.

Alternatively, the control circuitry may be located in the handle portion of the elongate device and configured to selectively connect respective conductive elements to one of first and second electrical connectors located on the handle portion, which in turn are connected to the respective first and second generator terminals. In either of these embodiments, the control circuitry allows for the selective configuration of the conductive elements forming the first and second electrodes, whether prior to—or during —a procedure using the electrosurgical device.

In one embodiment, an inner core of elements extends distally beyond an outer ring of elements, or vice versa, wherein each of the inner core and outer ring may selectively include one or more elements in each electrode. In one embodiment, the elements are positionable within a lumen of a delivery cannula, e.g., of an obtuator assembly, for locating the elements in a target tissue region in a body. In this or other embodiments, at least some of the elements may be biased to move radially outward in body tissue when the delivery cannula is retracted to expose the elements in the target tissue region. In another embodiment, the conductive elements have sufficient column strength, and are tethered together to collectively form a tissue piercing distal end of the elongate member, so as to allow the elongate member to be moved directly through solid body tissue for locating the elements in a target tissue region in a body.

In accordance with yet another aspect of the invention, a method of treating body tissue comprises providing a surgical device having a proximal handle portion and an elongate member extending distally from the handle portion, with a plurality of electrically conductive elements attached to a distal end of the elongate member, each element electrically insulated from the other elements. A first electrode comprising one or more of the conductive elements are electrically connected to a first terminal of a generator, and a second electrode comprising one or more further of the conductive elements are electrically connected to a second terminal of the generator. The distal end of the elongate member is positioned in a patient's body, so that the respective elements are located in a tissue region to be treated. Electrical energy is delivered through a circuit formed between the first and second generator terminals, the circuit including electrical conduction through tissue located between conductive element(s) of the first electrode and the conductive element(s) of the second electrode.

In one embodiment of this method, the conductive elements are located in the tissue to be treated by positioning the conductive elements within a lumen of a delivery cannula, locating a distal end of the delivery cannula in the tissue to be treated, and retracting the delivery cannula to expose the elements in the tissue. At least some of the elements may optionally be biased to move laterally outward into the tissue when the delivery cannula is retracted. In another embodiment, the elements have sufficient column strength and are tethered together to collectively form a tissue piercing distal end of the elongate member, so as to allow the elongate member to be moved directly through solid body tissue.

Other and further embodiments and aspects of the invention will become apparent when reviewing the following detailed description.

### BRIEF DESCRIPTION OF THE DRAWINGS

Various embodiments of the invention are disclosed in the following detailed description, and in the accompanying drawings, in which:

FIG. 1 is a partially cut-away side view of an electrosurgical device according to one embodiment of the invention, including a plurality of conductive elements attached at a distal end of the device;

FIG. 2 is a simplified schematic illustration of an electrosurgical system, including the electrosurgical device of FIG. 1, for treating a target tissue region in a patient;

FIG. 3 is an enlarged side view of the distal end portion of the electrosurgical device in FIG. 1;

FIG. 4 is a cross-sectional end view taken along line 4-4 in FIG. 3, illustrating the respective conductive element polarities according to one embodiment;

FIG. 5 is the cross-sectional end view shown in FIG. 4, illustrating the respective conductive element polarities according to an another embodiment;

FIGS. 6A-6C are side views of alternative conductive element arrangements for an electrosurgical device such as that shown in FIG. 2; and

FIGS. 7A-7B are partially cut-away schematic views of alternative handle portions for an electrosurgical device such as that shown in FIG. 2.

### DETAILED DESCRIPTION OF THE ILLUSTRATED EMBODIMENTS

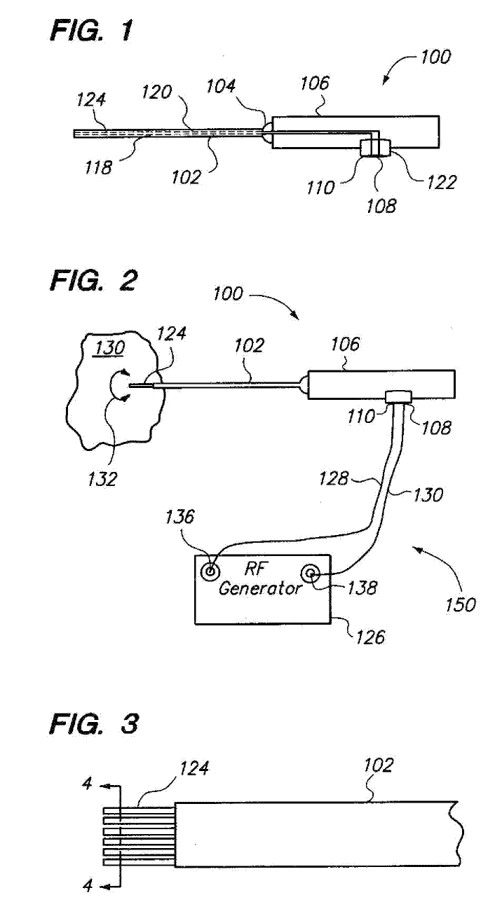

FIG. 1 illustrates an electrosurgical device 100 according to one embodiment of the invention, which generally includes an elongate member 102 attached via connector 104 to a handle 106, with a plurality of conductive elements 124 attached to, and extending distally from, a distal end of the elongate member 102 (best seen in FIG. 3). It will be appreciated that, in an alternate embodiment, the electrosurgical device 100 may be formed from a single-body construction, having a proximal handle portion and an elongate distal portion extending there from. The handle 106 includes a recessed connector housing 122, with first and second electrical connector pins 108 and 110 located therein. As indicated by dashed lines 120 and 118, connector 108 provides an electrical connection to a first electrode comprising one or more of the conductive elements 124, and connector 110 provides a separate electrical connection to a second electrode comprising one or more further of the conductive elements 124, and electrically isolated from the one or more conductive elements 124 of the second electrode.

FIG. 2 depicts an electrosurgical system 150, including device 100, while in use. In particular, the distal end of the elongate member 102 is inserted into the solid body tissue of a patient, so that the conductive elements 124 are located, e.g., using ultrasound localization, in a target tissue region 130 to be treated. The electrical connector pins 110 and 108 in the handle 106 of the device 100 are connected via cables 128 and 130 to respective first (e.g., “active”) and second (e.g., “return”) terminals 136 and 138 of a radiofrequency (RF) generator 126. The generator 126 is activated to supply power through an electrical circuit that is completed by electrical conduction (indicated by arrows 132) through the target tissue region 130 between the one or more conductive elements 124 of the first electrode and the one or more conductive elements 124 of the second electrode, causing ablation of the tissue proximate the conductive elements 124.

In various embodiments, the conductive elements 124 may be formed from conductive metal(e.g., stainless steel, titanium, and others), alloys, or other materials (e.g., polymers, Nitinol®, Inconel®) that have an impedance which is lower than the surrounding materials or tissue. Each of the elements 124 may be partially insulated (not shown), e.g., around a base portion, with only a distal end portion of the element being exposed. The one or more conductive elements 124 forming a first electrode are electrically connected to connector pin 108, and the one or more conductive elements 124 forming the second electrode are electrically connected to connector pin 110.

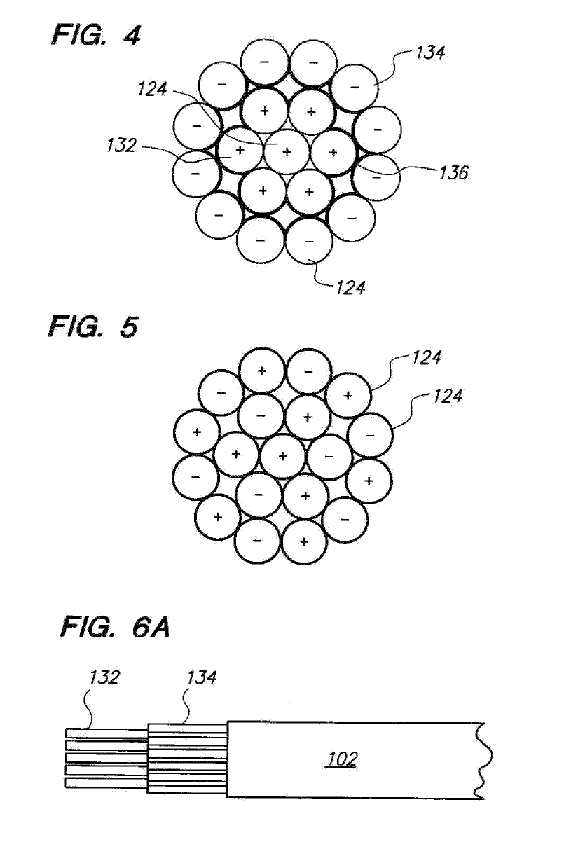

Referring to FIG. 4, in one embodiment, the first electrode comprises an inner core 132 of conductive elements 124, and the second electrode comprises an outer ring 134 of conductive elements 124 at least partially surrounding the inner core 132. In the illustrated embodiment, the conductive elements of the inner core 132 are isolated from the conductive elements of the outer ring 134 by an insulating material 136 interposed between the respective core and ring elements. The material 136 may extend distally co-extensive with the respective elements of one or both of the inner core 132 and outer ring 134, or less than (or even greater than) co-extensively.

It will be appreciated that the inner core 132 need not necessarily comprise any particular number of conductive elements 124, and that even a single element may comprise the entirety of the inner core 132 in some embodiments. Similarly, the outer ring 134 need not completely surround the inner core 132, and is also not required to comprise any particular number of elements 124. Further, the distal end of the elongate member 102 need not be circular, but may be any number of other cross-sectional geometries, e.g., triangular, rectangular, or asymmetrical.

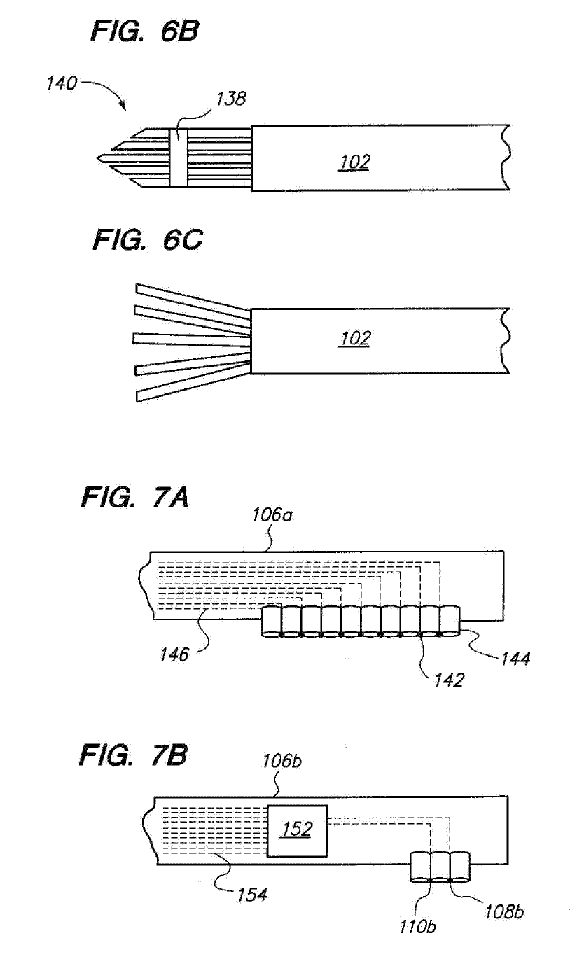

The length and/or out diameter of the respective elements 124 may be uniform or may vary, depending on the desired electrode pattern. By way of non-limiting example, FIG. 6A depicts an embodiment in which the conductive elements of the inner core 132 extend distally beyond the conductive elements of the outer ring 134. By way of another non-limiting example, FIG. 6B depicts the respective conductive elements 124 tethered together with an annular weld 138, with the element tips beveled to collectively form a tissue piercing distal tip 140 of the elongate member 102. This construction allows the elongate member 102 to be positioned directly through solid body tissue for locating the elements 124 in a target tissue region, while minimizing collateral tissue damage to the patient. In this or similar embodiments, the conductive elements 124 preferably have sufficient column strength to readily track through solid tissue without collapsing. In some embodiments, the elements 124 may be braided together or around a mandrel (not shown) to provide further structural integrity to facilitate smooth tracking through solid tissue. In all such embodiments, electrical isolation between elements of the respective electrodes is maintained.

The conductive elements may be arranged in a wide variety of patterns and configurations. By way of one example, the subset of elements forming the respective electrodes s may be divided into hemispheres of the elongate member distal end. By way of another example, the conductive elements of one electrode may be interleaved (but still electrically isolated from) with the elements of the other electrode, such as shown in FIG. 5, to provide maximum flexibility in forming possible electrode patterns. By way of yet another example, in an embodiment such as but not limited to that shown in FIG. 5, the conductive elements can be arranged in a pattern similar to that shown in FIG. 6A, but instead of the elements of the inner core 132 and outer ring 134 comprising the respective first and second electrodes, the first and second electrodes each include one or more elements of the inner core 132 and of the outer ring 134.

In one embodiment, the elongate member 102 and conductive elements 124 are positionable within a lumen of a delivery cannula (not shown), e.g., of an obtuator assembly, for locating the elements 124 in a target tissue region in a body, such as shown in system 150 of FIG. 2. This manner of locating the conductive elements 124 in the target tissue region is similar to that used by the LeVeen CoAccess product manufactured and distributed by Boston Scientific Corporation, and taught in U.S. Pat. No. 5,855,576, the contents of which are hereby fully incorporated herein by reference. One notable difference is that, in some embodiments of the present invention, the respective conductive elements may not have sufficient column strength, or tissue piercing tips. In such embodiments, rather than the elements being pushed through the tissue out of the open distal end of the introducer cannula (as with the LeVeen CoAccess), the introducer cannula is retracted axially relative to the elongate member 102, to expose the elements in contact with the surrounding tissue. In this and/or other embodiments, at least some of the conductive elements 124 may be biased to fan radially outward (as shown in FIG. 6C) in body tissue when the delivery cannula is retracted to expose the elements in the tissue.

It will be appreciated that a given conductive element 124 may be electrically connected, either directly or indirectly, to one or more other elements 124 that are part of the same electrode by using a weld, e.g., laser, braze, seam, spot, butt, and the like, which may also provide an electrical connection between the respective elements. In other embodiments, a weld may provide only structural connectivity between two or more elements. Each element 124 has an exposed surface area near its distal tip, providing for electrical contact with tissue where a high current density may be generated in order to cause cell necrosis (ablation) due to the current conduction through the tissue between respective elements of the first and second electrodes.

In accordance with one aspect of the invention, an electrosurgical system, such as system 150 shown in FIG. 2, further includes control circuitry which selectively electrically connects the one or more conductive elements 124 that form the first electrode to the first generator terminal 136, and the one or more conductive elements 124 that form the second electrode to the second generator terminal 138. In one embodiment, shown in FIG. 7A, each of the conductive elements carried on the elongate device is electrically connected to a respective electrical connector 142 of a multi-pin connector 144 located on the handle portion 106(a) of the device, as represented by the dashed lines 146. In this embodiment, the control circuitry (not shown) is located external to the elongate device 100(a), e.g., in an adjunct device (not shown) interposed in the circuit between the RF generator and the device, and is configured to electrically connect selected ones of the conductive elements to the first or second generator terminals via the respective electrical connectors 142. It will be appreciated that the number of connectors 142, and by extension the number of conductive elements 124, in the embodiment of FIG. 7A is for purposes of illustration only and, in alternate embodiments, the actual number may be greater or less than the number shown, depending on the desired electrode pattern.

Alternatively, as shown in FIG. 7B, the control circuitry 152 may be located in the handle portion 106(b) of the electrosurgical device and configured to selectively connect respective conductive elements (represented by dashed lines 154) to one of first and second electrical connectors 108(b) and 110(b) located on the handle portion 106(b). The first and second electrical connectors 108(b) and 110(b), in turn, are connected to the respective first and second generator terminals (not shown in FIG. 7B). As with the embodiment in FIG. 7A, the control circuitry 152 in the embodiment of FIG. 7B allows for the selective grouping of the conductive elements for customizing the first and second electrode patterns, whether prior to or during a procedure being performed using the electrosurgical device. Again, it will be appreciated that the number of conductive elements in the embodiment of FIG. 7B (represented by the dashed lines 154) is for purposes of illustration only and, in alternate embodiments, the actual number may be greater or less than the amount shown.

The control circuitry 152 may be implemented in hardware and/or software, and is controlled via a user interface located on the respective device housing the control circuitry. Preferably, the control circuitry 152 allows for the user to configure the electrode elements and/or to choose between some number of previously configured electrode patterns. In one embodiment, the control circuitry 152 automatically configures (or reconfigures) the respective electrode element subsets depending on the desired ablation pattern to be achieved and/or on other parameters, including real-time data monitored during a procedure, such as impedance or temperature data. The control circuitry 152 may also allow for the electrosurgical device to be operated in mono-polar mode, where all of the conductive elements are connected to the “active” terminal, and a conventional ground pad is connected to the “return” terminal.

Although the foregoing embodiments have been described in some detail for purposes of clarity of understanding, the implementation of these and other embodiments of the invention are not limited to the details and examples provided above.

What is claimed:

1. An electrosurgical device, comprising:
a handle portion having first and second electrical connectors; and
an elongate member extending from the handle portion and having a distal end portion carrying a plurality of electrically conductive elements, a first electrode comprising one or more of the conductive elements electrically connected to the first electrical connector, and a second electrode comprising one or more of the further conductive elements electrically connected to the second electrical connector, with the one or more elements of the first electrode being electrically insulated from the one or more elements of the second electrode,
wherein the first and second electrical connectors are adapted for connection to respective terminals of a power source to form an electrical circuit that is completed by electrical conduction between the first electrode and the second electrode.

2. The device of claim 1, the first electrode comprising an inner core of conductive elements, the second electrode comprising an outer ring of conductive elements at least partially surrounding the inner core.

3. The device of claim 1, wherein the one or more elements of the first electrode extend distally beyond the one or more elements of the second electrode.

4. The device of claim 1, wherein the elongate member and conductive elements are positionable within a lumen of a delivery cannula for locating the elements at a tissue region to be treated in a body.

5. The device of claim 4, wherein at least some of the conductive elements are biased to fan outward in body tissue when the delivery cannula is retracted to expose the elements in the tissue region.

6. The device of claim 1, wherein the conductive elements have sufficient column strength and are tethered sufficiently together with their distal tips collectively forming a tissue piercing distal end of the elongate member so as to allow the elongate member to be moved through body tissue.

7. An electrosurgical system, comprising:
a generator including first and second terminals for forming an electrical circuit;
an elongate device having a proximal handle portion and an elongate distal end portion, the distal end portion carrying a plurality of electrically conductive elements, each element electrically insulated from the other elements; and
control circuitry which selectively electrically connects a first electrode comprising one or more of the elements to the first generator terminal and a second electrode comprising one or more of the further elements to the second generator terminal to form an electrical circuit that is completed by electrical conduction between the first electrode and the second electrode.

8. The system of claim 7, wherein each conductive element is connected to a respective electrical connector located on the handle portion of the elongate device.

9. The system of claim 8, wherein the control circuitry is located external to the elongate device and is configured to selectively electrically connect respective ones of the conductive elements to one of the first and second generator terminals via the respective electrical connectors.

10. The system of claim 9, wherein the control circuitry is located in the generator.

11. The system of claim 7, wherein the control circuitry is located in the handle portion of the elongate device and coupled to first and second electrical connectors located on the handle portion, the first and second electrical connectors connected to the respective first and second generator terminals.

12. The system of claim 7, wherein the distal elongate portion and conductive elements are positionable within a lumen of a delivery cannula for locating the elements at a tissue region to be treated in a body.

13. The system of claim 12, wherein at least some of the conductive elements are biased to fan outward in body tissue when the delivery cannula is retracted to expose the elements in the tissue.

14. The system of claim 7, wherein a group of inner conductive elements extend distally beyond a group of outer conductive elements.

15. The system of claim 14, the first and second electrodes each including one or more elements from each of the inner group and the outer group.

16. A method of treating body tissue, comprising:
providing an elongate device having a proximal handle portion and a distal end portion, the distal end portion carrying a plurality of electrically conductive elements, each element electrically insulated from the other elements;
electrically connecting a first electrode comprising one or more of the elements to a first terminal of a generator;
electrically connecting a second electrode comprising one or more of the further elements to a second terminal of a generator;
positioning the distal end portion of the elongate device in a body, so that the respective elements of the first and second electrodes are located adjacent tissue to be treated; and
delivering electrical energy through a circuit formed between the first and second generator terminals, the circuit including electrical conduction through tissue located between the one or more elements of the first electrode and the one or more elements of the second electrode.

17. The method of claim 16, wherein positioning the distal end portion of the elongate device includes
positioning the conductive elements within a lumen of a delivery cannula;
locating the delivery cannula proximate the tissue to be treated; and
retracting the delivery cannula to expose the conductive elements in the tissue.

18. The method of claim 17, wherein at least some of the conductive elements are biased to fan outward into the tissue when the delivery cannula is retracted.

19. The method of claim 16, wherein a group of inner conductive elements extend distally into the tissue beyond a group of outer conductive elements.

20. The method of claim 19, the first and second electrodes each including one or more conductive elements from each of the inner group and the outer group.
